(19) United States
(12) Patent Application Publication
(10) Pub. No.: US 2005/0053549 A1
(43) Pub. Date: Mar. 10, 2005

(54) Method for treating airway disorders

(75) Inventors: Rajiv Parikh, Palo Alto, CA (US); Bhairavi Parikh, Palo Alto, CA (US); Andrew Newman, Palo Alto, CA (US)

Correspondence Address: DANIEL P. MAGUIRE, 423 E ST., DAVIS, CA, 95616, US

(73) Assignee: Aperon Biosystems Corp.

(21) Appl. No.: 10/659,408

(22) Filed: Sep. 10, 2003

(51) Int. Cl. A61K 49/00; A61B 5/00

(52) U.S. Cl. 424/9.1; 600/300

(57) ABSTRACT

A method for management of asthma and other respiratory conditions is disclosed. This method uses exhaled nitric oxide readings to measure airway inflammation, thereby allowing precise titration of medication. The present invention can also be used to measure patient compliance with prescribed treatment regimes.

### BACKGROUND OF THE INVENTION

1. Field of the Invention

This invention relates to methods for treating airway disorders such as asthma.

2. General Background

Asthma

Asthma is a chronic condition in which allergens or other triggers cause changes in a subject's airways, resulting in coughing, wheezing, and shortness of breath (dyspnea). There are two stages in an asthmatic attack: the hyperreactive stage, and the inflammatory stage. In the hyperreactive stage, inhaled allergens or other irritants cause smooth muscles in the airways to excessively constrict and narrow. In the inflammatory stage, the immune system responds to the allergens or other stimuli by delivering white blood cells and other immune factors to the airways. These factors cause the airways to swell, to fill with fluid, and to produce a thick sticky mucous. This immune response causes wheezing, breathlessness, an inability to exhale properly, and a phlegm-producing cough.

Inflammation is present in the lungs of all patients with asthma, even when they are not experiencing symptoms. When the airways become inflamed, the body responds by releasing nitric oxide into the local environment presumably to induce local pulmonary vasodilation.

Management and Monitoring of Asthma

Managing asthma is an ongoing challenge for clinicians, in large part because it has been difficult to accurately assess a patient's asthmatic status. Currently, physicians attempt to monitor asthma severity through clinical exam, pulmonary function testing (PFT), and peak flow meter measurements.

However, these tests provide only a relatively crude tool for asthma management. For instance, one PFT measurement, the FEV1 test (forced expired volume in one second), is not sensitive enough to effectively manage mild cases of asthma. Likewise, the PC20 (provocative concentration causing a 20% fall in FEV1) test is affected by corticosteroids, and therefore cannot be routinely performed in asthmatics who take such medications. Both the FEV1 and PC20 parameters are slow to change, and cannot distinguish the effects of steroid dosages. Indeed, no traditional asthma monitoring technique is sensitive enough to indicate dose-dependent effects of inhaled steroids or other medications. It is important to titrate corticosteroids due to potential unwanted side effects in adults and children.

Other conventional asthma management tests also have their shortcomings. For instance, studies have shown that peak flow meter measurements are unreliable and inadequate.

The fundamental problem with these traditional asthma monitoring techniques is their inability to directly measure airway inflammation. Given the shortcomings of the current asthma management techniques, there is a need for new procedures that can be tied more directly to airway inflammation. The present invention, as described below, provides such techniques.

### SUMMARY OF THE INVENTION

The present invention is a method for managing asthma using exhaled nitric oxide (“eNO”). More particularly, the present invention can be used by clinicians to precisely titrate asthma medication based on the level of inflammation in a subject's airways. The present invention can also be used to ensure that asthma patients comply with their treatment regimes.

### BRIEF DESCRIPTION OF THE DRAWINGS

FIG. 1 is a flowchart depicting the basic process of managing asthma based on eNO readings, according to an embodiment of the present invention.

FIG. 2 is a flowchart depicting a management protocol for a patient with mild persistent asthma, according to an embodiment of the present invention.

FIG. 3 is a flowchart depicting a management protocol for a patient with moderate asthma, according to an embodiment of the present invention.

FIG. 4 is a flowchart depicting a management protocol for a patient with severe asthma, according to an embodiment of the present invention.

### DETAILED DESCRIPTION

The present invention is a method of managing asthma and other airway disorders using eNO values. To practice this invention, a sensing device for measuring eNO is needed. Suitable devices for this purpose include: Aerocrine NIOX, Sievers Nitric Oxide Analyzer (NOA 280i), Ekips Breathmeter or the ECO PHYSICS CLD 88sp with Exhalyzer D.

A particularly suitable device for practicing the present invention is described in the U.S. patent application with Ser. No. 10/334,625. The disclosure of that application is incorporated by reference herein, as if set out in full. Combination devices that include both a nebulizer and an eNO measurement sensor can also be used.

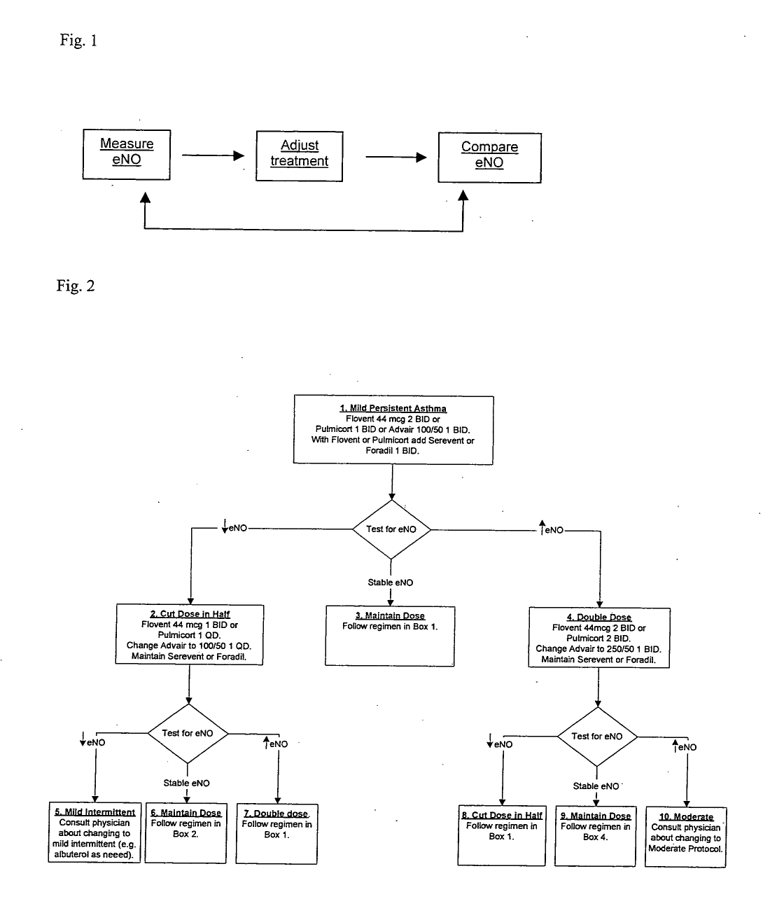

The basic process of the present invention is presented in FIG. 1. Under this process, the first step is measuring a subject's exhaled NO. This measurement could be a single measurement at a point in time or a series of measurements over a period of time. The frequency of this measurement depends on the goal of the monitoring. As described below, in one embodiment the subject would check his or her eNO level once per day, such as every morning.

The second step in the process is comparison of the subject's eNO value to a function, threshold, range, or curve. Of course, the nature of the function, threshold, range, or curve will vary depending on the purpose of the monitoring. For this patent, the term “function” shall be used to generically denote any function, range, threshold, curve, table, or schedule that relates measured eNO values to changes in the subject's treatment protocol or compliance activity. “Function” includes any sort of algorithm or method that relates eNO values with changes in treatment or compliance behavior, and includes both formal or mathematical correlation structures, as well as informal or even unwritten, intuitive methods.

If the monitoring is for adjusting anti-inflammatory medication, then the measured value can be compared to a curve that correlates eNO to medication levels. Such curves can be tailored for each patient and each medication, and can have ranges which indicate a “normal” level of eNO, an “under control” level, and an “elevated” level.

If the process is being used to ensure patient compliance, the measured eNO can be compared to a predetermined baseline that is established for the subject at the start of therapy or based on normative patient data. The eNO readings can also be compared to the ranges that indicate whether a subject has normal or elevated eNO levels. Exhaled nitric oxide levels are expected to deviate with the course of interventional therapy. It is generally expected that steroid delivery will decrease the amount of exhaled NO over time, and thus physicians can use eNO concentrations to determine if the patient is complying with a prescribed treatment protocol.

The third step in the process is management of the subject's condition based on the comparison of the measured value to the applicable threshold, range, or curve. For instance, if the reading indicates that the subject's eNO level is elevated, his or her medication can be incrementally increased in accordance with a predetermined function. Or if the eNO readings suggest that a subject is not complying with his or her treatment regime, an appointment with a clinician can be made to encourage future compliance. Other possible interventions include changing the type of medication, modifying the patient's activity level, changing environmental exposure to triggers, increasing or decreasing vigilance with symptoms or changing the frequency of physician visits.

Various medications can be used with the present invention, including but not limited to the following: (i) inhaled corticosteroids, such as Flovent (fluticasone propionate), Pulmicort (Budesonide), and QVAR (beclomethasone dipropionate), (ii) leukotrience receptor agonists, such as Singular (montelukast) and Accolate (zafirlukast), (iii) long acting beta-2 agonists, such as Serevent (salmeterol) and Foradil (formoterol), and (iv) combination therapies such as Advair (fluticasone propionate and salmeterol). Of course, other anti-inflammatory medications can be used with the present invention, and the list provided above is only illustrative. Also, the medications used in the present invention can be administered in any medically acceptable manner, including orally, intravenously, or transdermally, or through inhalers, nebulizers, etc.

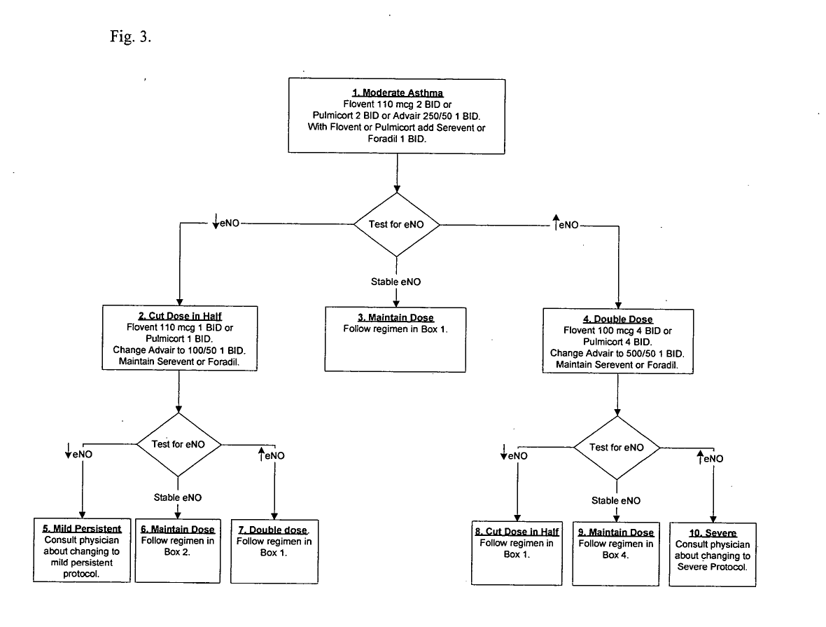

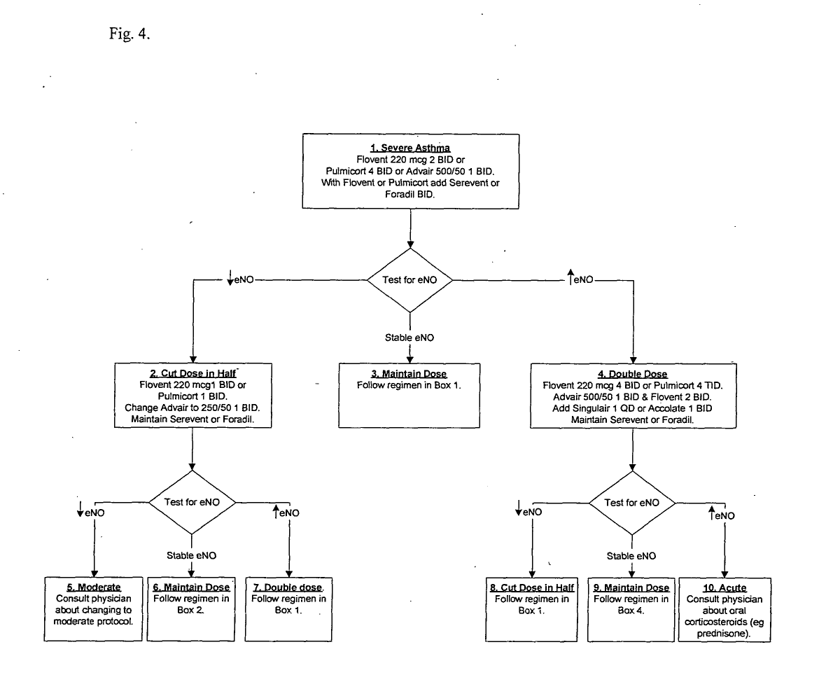

The process of the present invention may be adjusted based on the severity of the patient's asthma. The NIH has established guidelines for classifying asthmatics as having either “mild intermittent,” “mild persistent,” “moderate persistent,” or “severe persistent.” The protocol for adjusting medication based on exhaled nitric oxide reading may vary based on the severity of the patient's asthma. Thus, FIG. 2 shows a sample decision tree for patients with mild persistent asthma, FIG. 3 shows a sample decision tree for patients with moderate persistent asthma, and FIG. 4 shows a sample decision tree for patients with severe persistent asthma. Each of these decision trees was designed for adults, and the protocol for children would have lower dosages. In each of these protocols, it is assumed that the patient has been initially prescribed one or more anti-inflammatory medications by his or her physician. However, the present invention can also be used to establish an initial medication regime for a newly diagnosed asthmatic.

In these sample protocols, the eNO measurement frequency would vary with the severity of the condition and based on physician recommendation. For instance, patients with severe or volatile conditions would measure themselves daily, while patients with moderate symptoms would take readings 2 to 3 times a week, and those with mild asthma may measure their eNO only seasonally or sporadically. The patient's data would then be provided to the physician, who would titrate medications on a yearly, quarterly, monthly or weekly basis. Alternatively, if a combination device as described above is used, the built-in nebulizer can automatically adjust the patient's medication.

For each of the protocols described in FIGS. 2, 3, and 4, if a patient is prescribed medication after receiving an initial diagnosis of asthma, then the patient would follow the physician's initial recommendations for at least seven days before titrating based on eNO readings. After the initial seven days, if the patient has an eNO measurement that is trending downward, but not in the stable range, then the initial regimen should be continued. In each case, the patient would test eNO levels periodically, such as every day, every two to three days, once per week, etc. If the dosage is changed, it should be maintained for five to seven days before changing again.

In these figures, the following abbreviations are used: “QD”: 1 time daily, “BID”: 2 times daily, “TID”: 3 times daily, “QID”: 4 times daily, “2 BID”: 2 puffs of medication, 2 times a day or 4 puffs once per day, and “4 QID”: 4 puffs, 4 times a day or 16 puffs once per day. When the phrase “Stable eNO” is used in these figures, it refers to an exhaled nitric oxide range between 20-30 ppb, measured at a 50 ml/sec flow rate. (Other flow rates, such as 250 ml/sec, can also be used, with concomitant adjustment of the eNO levels). The legend “teNO” refers to an increase of ≧5 ppb of exhaled nitric oxide from the stable eNO range. The legend “↓eNO” refers to a decrease of ≧5 ppb exhaled nitric oxide from the stable eNO range. The x/y ratio provided for the medication for Advair refers to x mcg of fluticasone propionate and y mcg salmeterol per dose. See FIG. 3.

For each of these protocols, if the measured eNO level is over 75 ppb, measured at 50 ml/sec, then the patient should consult his or her physician.

The overall objective of the titration systems is to maintain the patient in a stable eNO range, as defined above.

The protocols of FIGS. 3, 4, and 5 are only illustrative, and physicians can create their own treatment plans without departing from the spirit or scope of this patent. For instance, a physician may decide that his severe asthma patient should use a modified version of the “moderate” protocol of FIG. 4, adding Singulair 1 BID to the regimen. The physician might also redefine the stable range to be from 20 ppb to 40 ppb, or might request that the patient contact the physician if there is a 10 ppb increase in a 3 day period.

The present invention offers a number of advantages over prior art management techniques. For patients, the benefits include (i) reductions in asthmatic attacks, as a result of better monitoring and management, (ii) avoidance of the long term airway remodeling that results from uncontrolled asthma, (iii) avoidance of the short and long term side effects from overmedication with anti-inflammatory agents, (iv) reduction in the need for short term beta agonist or rescue inhalers, and (v) reduction in the need for long term beta agonists and oral corticosteroids.

For physicians, the present invention allows more effective asthma management, because the physician has much better information about the patient's condition and can make more efficacious treatment decisions. Specifically, using the process outlined in the present invention, physicians can determine the actual level of airway inflammation in their patients at any given time, or over a period of time based on periodic measurements and make more appropriate treatment decisions. Physicians can also adjust treatment based on the inflammation effect of various triggers, including environmental conditions, as well as the inflammation effects of viral, bacterial and/or respiratory conditions. The present invention additionally allows clinicians to accurately evaluate the comparative effects of various anti-inflammatory therapeutics, and to optimize the doses of a particular medication or a set of medications. Finally, the present invention makes it much easier for physicians to increase the level of compliance with prescribed treatment programs.

For payors and society at large, the present invention will help reduce the direct and indirect costs of asthma. Better asthma management resulting from this invention means fewer emergency room visits, more efficient use of medication, and less resources spent treating asthma attacks.

Although it is well-suited for management of asthma, the present invention can also be used to treat other airway-based disorders that are treated with anti-inflammatory medications, such as chronic bronchitis, lupus, or cystic fibrosis. Nitric oxide is a generic marker for inflammation, so the present invention can be used for virtually any airway inflammation condition.

Additionally, the present invention includes management of respiratory conditions using other exhaled gases besides eNO or combinations thereof. For instance, other potential exhaled gases include: carbon monoxide, acetone, and hydrocarbons such as ethane and penthane. Additionally, the present invention can be used to manage conditions based on the evaluation of exhaled breath condensate analytes such as hydrogen peroxide, 8-Isoprostane, 3-Nitrityrosine, leukotriene B4 and cysteinyl-leukotrienes, prostaglandins, histamine, adenosine, cytokines (interleukin-4, interferon-γ), pH.

Although the present invention can be used in a number of different texts and for a number of different purposes, specific prophetic examples are vided below.

### EXAMPLE 1

This example, a patient with “mild persistent” asthma tests his or her eNO weekly and generally follows the protocol of FIG. 2. The table below provides an exemplary timeline, with eNO readings and responsive treatments. “Box” references are to FIG. 2.

| | eNO | |
|---|---|---|
| Day | (ppb) | Treatment |
| 1 | 55 | Pulmicort 1 BID and Serevent 1 BID for 7 days (Box 1) |
| 7 | 50 | Increase to to Pulmicort 2 BID (Box 4). Maintain Serevent |
| | | 1 BID throughout. |
| 14 | 25 | Stay on course, Stable Range |
| 21 | 20 | Stay on course, Stable Range |
| 28 | 12 | Decrease to Pulmicort 1 QD (Box 2) |
| 35 | 21 | Stay on course, Stable Range |
| 42 | 16 | Stay on course, though outside Stable Range, not ≧5 ppb |
| | | below. |
| 49 | 12 | Contact physician. Stop Pulmicort and Serevent. Take |
| | | Albuterol as needed. Restart on Box 2 regimen if higher |
| | | than Stable Range. |
| 56 | 20 | Stay on course, Stable Range |
| 63 | 25 | Stay on course, Stable Range |
| 70 | 31 | Take Pumicort 1 QD and Serevent 1 BID |

### EXAMPLE 2

This example, a patient with “moderate” asthma tests his or her eNO every other day, and generally follows the protocol of FIG. 3. The table below provides an exemplary timeline, with eNO readings and responsive treatments. “Box” references are to FIG. 3.

| | eNO | |
|---|---|---|
| Day | (ppb) | Treatment |
| 1 | 60 | Flovent 110 mcg 2 BID and Serevent |
| | | 1 BID for 7 days (Box 1) |
| 3 | 40 | Stay on course, appropriate trend |
| 7 | 30 | Stay on course, in Stable Range |
| 15 | 42 | Increase to Flovent 110 mcg 4 BID for at least 1 week. |
| | | Continue Serevent 1 BID throughout (Box 4). |
| 19 | 38 | Stay on course, appropriate trend. |
| 23 | 25 | Stay on course, in Stable Range |
| 45 | 13 | Decrease to Flovent 110 mcg 2 BID for at least 1 week |
| | | (Box 8 which refers back to Box 1) |
| 55 | 25 | Stay on course, in Stable Range |
| 63 | 38 | Increase to Flovent 110 mcg 4 BID for at least 1 week (Box |
| | | 4). |
| 71 | 43 | Contacts physician and moves to Severe Protocol Box 1. |
| | | Takes Flovent 220 mcg 2 BID. If rise continues, move up |
| | | dose. |
| 75 | 48 | Increase to Flovent 220 mcg 4 BID |
| 79 | 40 | Stay on course, appropriate trend |
| 83 | 35 | Stay on course, appropriate trend |
| 87 | 29 | Stay on course, in Stable Range |

### EXAMPLE 3

This example, a patient with “severe” asthma tests his or her eNO every day, and generally follows the protocol of FIG. 4. The table below provides an exemplary timeline, with eNO readings and responsive treatments. “Box” references are to FIG. 4.

| | eNO | |
|---|---|---|
| Day | (ppb) | Treatment |
| 1 | 80 | Advair 500/50 1 BID (Box 1) |
| 3 | 72 | Stay on course, appropriate trend |
| 7 | 50 | Stay on course, appropriate trend |
| 10 | 42 | Stay on course, appropriate trend |
| 14 | 30 | Stay on course, Stable Range |
| 25 | 25 | Stay on course, Stable Range |
| 31 | 37 | Increase dose by adding Flovent 220 mcg 2 BID and |
| | | Singulair 1 QD for 7 days (Box 4). |
| 33 | 33 | Stay on course, appropriate trend |
| 37 | 28 | Stay on course, Stable Range |
| 48 | 23 | Stay on course, Stable Range |
| 55 | 42 | Contact physician. Increase dose by adding Prednisone |
| | | 60 mcg for 3 days only. Contact physician is eNO |
| | | continues to rise (Box 10 and return to Box 4)) |
| 56 | 30 | Stay on course, appropriate trend. Prednisone should be |
| | | dropped. |
| 59 | 25 | Stay on course, Stable Range |
| 68 | 15 | Decrease dose by dropping use of Singulair and Flovent. |
| | | Continue with Advair 500/50 1 BID and monitor for 7 |
| | | days (Box 8 which returns to Box 1) |
| 76 | 13 | Decrease dose to Advair 250/50 1 BID for 7 days (Box 2). |
| 83 | 20 | Stay on course, Stable Range |
| 88 | 15 | Contact physician - recommends continuing dose, but as |
| | | part of Moderate Asthma Protocol (Box 5 to Box 1 |
| | | Moderate Asthma Protocol) |
| 93 | 10 | Change to Advair 100/50 1 BID (Box 2) |

### EXAMPLE 4

In this example, a patient with “moderate” to “severe” asthma does not have access to a home eNO measurement system, so the patient visits his or her doctor every month for monitoring. The table below provides an exemplary timeline, with eNO readings and responsive treatments. “Box” references are to FIGS. 3 or 4, as indicated.

| | eNO | |
|---|---|---|
| Day | (ppb) | Treatment |
| 1 | 80 | Flovent 220 mcg 2 BID and Serevent 1 BID (Box 1 of FIG. |
| | | 4. Maintain Serevent 1 BID throughout |
| 30 | 32 | Stay on course, slightly outside of Stable Range |
| 60 | 25 | Stay on course, Stable Range |
| 90 | 50 | Increase to Flovent 220 mcg 4 BID (Box 4 of FIG. 4) |
| 120 | 15 | Decrease to Flovent 2 BID (Box 8 refers back to Box 1 of |
| | | FIG. 4) |
| 150 | 13 | Decrease to Flovent 1 BID (Box 2 of FIG. 4) |
| 180 | 18 | Stay on course, Stable Range |
| 210 | 10 | Contact Physician (Box 5). Change to Flovent 110 mcg 2 |
| | | BID (Box 1 of FIG. 3). |
| 240 | 12 | Decrease to Flovent 110 mcg 1 BID (Box 2 of FIG. 3) |
| 270 | 22 | Stay on course, Stable Range |
| 300 | 36 | Increase to Flovent 110 mcg 2 BID (Box 7 of |
| | | FIG. 3 to Box 1 of FIG. 3 regimen) |

### EXAMPLE 5

This example, a diagnosed asthmatic with questionable compliance habits is given an eNO measurement device, such as the device of Ser. No. 10/334,625. The subject is told to take eNO measurements once a day for 1 week and once a month thereafter., The device can hold at least 20 data points worth of data in its memory or storage. After each measurement, the data is transferred to the clinician' office for verification and review. The data can be transferred in a number of ways, including swapping of devices at the clinician's office, telephone or facsimile transmission, or direct uploading via the internet through a USB or COM port in the device.

The clinician would then review the data to determine compliance, as well as to titrate medications as described above. The clinician might infer noncompliance if the subject is consistently above the Stable Range or above 35 ppb. If the patient is over 70 ppb, the physician's office would contact the patient to take more immediate steps like prescribing a 3 to 7 day dose of Prednisone (oral corticosteroids).

One skilled in the art will appreciate that the present invention can be practiced by other than the preferred embodiments, which are presented for purposes of illustration and not of limitation.

1.) A method for managing a respiratory condition in a subject with a pre-existing treatment protocol, comprising:
causing measurement of an analyte in a subject's exhaled breath;
using said measurement in a function to determine changes to said existing treatment protocol; and
altering said treatment protocol in accordance with said changes.

2.) The method according to claim 1, wherein said respiratory condition is asthma.

3.) The method according to claim 2, wherein said analyte is nitric oxide.

4.) The method according to claim 3, wherein said step of altering said treatment protocol in accordance with said changes comprises changing the frequency of administering a medication.

5.) The method according to claim 3, wherein said medication is inhaled.

6.) The method according to claim 3, wherein said step of altering said treatment protocol in accordance with said changes comprises changing the dosage of a medication.

7.) The method according to claim 3, wherein said step of altering said treatment protocol in accordance with said changes comprises adding an anti-inflammatory medication in addition to those anti-inflammatory medications already part of said existing treatment protocol.

8.) The method according to claim 3, wherein said step of altering said treatment protocol in accordance with said changes comprises removing an anti-inflammatory medication from those anti-inflammatory medications that are part of said existing treatment protocol.

9.) The method according to claim 1, 2, 3, 4, 5, 6, 7, or 8 wherein said measurement occurs at least weekly.

10.) The method according to claim 9, wherein said measurement occurs at least daily.

11.) The method according to claim 10, additionally comprising uploading data from said measurement of the subject's exhaled nitric oxide concentration to a clinician using the internet.

12.) A method for ensuring that a subject with a respiratory condition follows a prescribed treatment protocol, comprising:
causing measurement of the subject's exhaled nitric oxide concentration to occur; and
using said nitric oxide concentration in a function to determine if the subject is following said prescribed treatment protocol.

13.) The method according to claim 12, wherein said respiratory condition is asthma.

14.) The method according to claims 13, wherein said measurement occurs at least weekly.

15.) The method according to claim 14, wherein said measurement occurs daily.

16.) A method for managing a respiratory condition in a subject, comprising:
causing measurement of the subject's exhaled nitric oxide concentration to occur; and
using said nitric oxide concentration to create a treatment protocol for said subject.

17.) The method according to claim 16, wherein said respiratory condition is asthma.
